(19) United States
(12) Patent Application Publication
(10) Pub. No.: US 2007/0214010 A1
(43) Pub. Date: Sep. 13, 2007

(54) SYSTEMS AND METHODS FOR UTILIZING A SECURE ELECTRONIC GATEWAY AT A PHYSICIAN'S OFFICE

(75) Inventors: Stephen J. Beaver, Columbus, OH (US); Lonnie W. Hardin, Laguna Niguel, CA (US); Eric D. Arnson, New Albany, IN (US); John G. Lettko, Orinda, CA (US)

Correspondence Address: SUTHERLAND ASBILL & BRENNAN LLP, 999 PEACHTREE STREET, N.E., ATLANTA, GA, 30309, US

(73) Assignee: ProxyMed, Inc., d/b/a MedAvant Healthcare Solutions, Atlanta, GA (US)

(21) Appl. No.: 11/621,476

(22) Filed: Jan. 9, 2007

Related U.S. Application Data
(60) Provisional application No. 60/757,255, filed on Jan. 9, 2006.

(51) Int. Cl. G06Q 10/00; A61B 5/00

(52) U.S. Cl. 705/2; 600/300

(57) ABSTRACT

A method of communicating with a physician's office that includes a host computer receiving, via the network interface, one or more data files from a data delivery device remotely located in a physician's office. The method further includes determining a set of data processing programs to be utilized when processing the received data files, where the determination is made based on a subscription associated with that particular physician's office. The method also includes processing the received data files utilizing at least a portion of the set of data processing programs, where the processing includes collecting or manipulating data files.

### CROSS-REFERENCE TO RELATED APPLICATION

This application claims the benefit of priority to U.S. Provisional Patent Application Ser. No. 60/757,255 entitled, “Systems and Methods for Utilizing a Secure Electronic Gateway at a Physician's Office,” which was filed in the United States Patent and Trademark Office on Jan. 9, 2006, the specification of which is hereby incorporated by reference.

### FIELD OF THE INVENTION

This invention relates to the secure communication of data between a physician's office and a variety of outside entities.

### BACKGROUND OF THE INVENTION

Various entities including laboratories, insurance companies, pharmacies, pharmaceutical companies, vendors, transcription companies, etc. communicate information—medical, financial or otherwise—with physician's offices through a wide variety of communication mediums including through a phone line, network, wireless, etc. Many of these entities employ a variety of communication protocols that must be adhered to at the physician's office. Such adherence can monopolize a physician's office's staff and communication resources (e.g., fax machines, computers, multi-function printers, bandwidth consumption, etc.). In some cases, a majority of a physician's office staff's time may be spend answering phone calls from third party entities, responding to fax requests from third party entities, downloading electronic files received from third party entities, uploading received data to a physician management system (PMS), printing, copying and/or forwarding electronically sent information, or inputting the received data into a user interface of the PMS system or electronic medical record (EMR) system manually for updating and storing the received information.

Additionally, much of the information received and transmitted from a physician's office includes confidential medical data including, for example, various types of laboratory results that are necessary for proper diagnosis of a doctor's patients, including blood work, DNA reporting, reviewing X-rays, tissue analysis as well as other medical data which is used in the treatment of patients. However, even today, the most common form of transferring this type of sensitive data is by way of a telephone line connected to a modem, which is in turn connected to a printer. The information is transmitted from the modem to the printer and then printed for review by a physician or other medical professional. This method of “send and hope” communication has no safeguards relating to privacy, security, accuracy and accountability for the sensitive data it transmits, particularly once the information is printed at the physician's office.

More recently, with the enactment of federal laws in the United States aimed at increasing the protection of patient privacy, these dedicated transmission devices have incorporated some forms of minimal error-checking (such as checking to see that all report fields have been completed such as the date, patient identification, serial number of the report, etc.) and security features including the requirement of the entry of a password or code to begin a transmission or a print operation. In these systems, if the error-checking conducted by the system detects no errors, a message is sent back to the sending party stating that the data was successfully delivered and the system would then disconnect from the physician's office. However, if the error-checking detects an error or potential error, then the transmission is halted and a message is returned to the sending party where the transmission originated, informing the sending party of the potential error and requesting resubmission once the error has been addressed.

However, these systems still leave many problems with secure medical data transmission to both physician's offices and outside entities unaddressed. One problem with the present means for achieving this form of data communication is that there are still significant gaps in reliability, detection, and accountability for data transmissions that result in errors, delivery to the incorrect location, or no delivery altogether. Moreover, current systems are still lacking in security features, which are especially important given the level of importance and private nature of the much of the data that is being transmitted. Current systems also fail to provide any meaningful remote system configurability, which would allow for remote maintenance, upkeep and troubleshooting to eliminate system downtime.

Further, as communication technology continues to lower the cost and complexity required to transfer large amounts of data in user-friendly formats, the data being transmitted has become larger and more complex. Many outside entities want to convey their information sent to a physician's office in a more informative, more attractive, and in some cases, more patient friendly ways through the use of color images, graphs, charts, figures or even multimedia presentations (e.g., video images or audio presentations of data, etc.). However, using current systems, an increase in the size and complexity of the data delivered may significantly lengthen the transmission time, which is generally undesirable.

For all the above stated reasons, current physician office communication systems lack connectivity options which would easily integrate and keep pace with new communication technology and new medical data processing devices which are increasingly being used in hospitals, doctor's offices, and laboratories. Thus, a need exists for a more secure, efficient, and reliable means for transmitting laboratory data to hospitals and doctor's offices, which addresses the shortcomings of the prior art listed above as well as the wants and needs medical professionals have in this area of secure data transmission.

### SUMMARY OF THE INVENTION

According to an embodiment of the invention, there is disclosed a method of communicating with a physician's office that includes a host computer receiving, via a network interface, one or more data files from a data delivery device located in a physician's office. The method further includes determining a set of data processing programs to be utilized when processing the received data files, where the determination is made based on a subscription associated with that particular physician's office. The method also includes processing the received data files utilizing at least a portion of the set of data processing programs, where the processing includes collecting and/or manipulating data files.

In accordance with one aspect of the invention, the method further includes receiving, via one or more input/output (I/O) interfaces, additional data files. According to another aspect of the invention, the method further includes transmitting, via the network interface, reconfiguration data to the data delivery device. In accordance with another aspect of the invention, the process of manipulating one or more data files includes formatting the data files. According to yet another aspect of the invention, the process of manipulating one or more data files includes tagging at least one data element for insertion into a report template. In accordance with another aspect of the invention, the method, further includes transmitting one or more tagged data elements via the network interface. According to yet another aspect of the invention, the method further includes upon tagging one or more data elements, inserting the data elements in the report template.

According to another embodiment of the invention, there is disclosed a data delivery device associated with a physician's office that includes a memory, where the memory contains one or more data processing programs and report templates. The data delivery device further includes a web server connected to a network, where the web server is accessible via a web browser, and where the web server is located behind a firewall at the physician's office. The data delivery device also includes a processor, in communication with the memory and web server. The processor is configured to execute software instructions for receiving one or more data files via the web browser, reformatting the data file(s) to a format readable by the data processing program(s), and processing the data file(s) utilizing the at least one processing program, where the processing includes extracting data from the data file(s) and populating one or more report templates with at least a portion of the extracted data.

In accordance with one aspect of the invention, the processor is configured to execute additional software instructions for storing the received data file in the memory. According to another aspect of the invention, the processor is configured to execute additional software instructions for transmitting the report template(s) to the web server. In accordance with yet another aspect of the invention, the memory includes user-defined parameters, and the processor is configured to execute additional software instructions for customizing the report template(s) based at least in part on the user-defined parameters.

According to another aspect of the invention, the data delivery device further includes one or more input/output (I/O) interfaces in communication with the processor, where the processor is configured to execute additional software instructions for transmitting the report template(s) to an external device. In accordance with yet another aspect of the invention, the data delivery device further includes one or more input/output (I/O) interfaces in communication with the processor, where the I/O interface(s) is in communication with a practice management system associated with the physician's office and/or an electronic medical records system associated with the physician's office.

According to yet another embodiment of the invention, there is disclosed a host computer that includes a memory, where the memory contains data processing programs. The host computer further includes a network interface in communication with a network. The host computer also includes a processor, in communication with the memory and network interface. The processor is configured to execute software instructions for receiving, via the network interface, one or more data files from a data delivery device located in a physician's office remote from the host computer. The processor further is configured to execute software instructions for determining a set of data processing programs to be utilized when processing the data file(s), where the determination is made based on a subscription associated with the physician's office. The processor also is configured to execute software instructions for processing the data file(s) utilizing at least a portion of the determined set of data processing programs, where the processing includes collecting and/or manipulating the data file(s).

In accordance with one aspect of the invention, the host computer includes one or more input/output (I/O) interfaces in communication with the processor, where the processor is configured to execute additional software instructions for receiving, via the I/O interface(s), additional data files. According to another aspect of the invention, the processor is configured to execute additional software instructions for transmitting, via the network interface, reconfiguration data to the data delivery device. In accordance with another aspect of the invention, the software instructions for manipulating the at least one data file include formatting the at least one data file.

According to yet another aspect of the invention, the software instructions for manipulating the at least one data file include tagging at least one data element for insertion into a report template. In accordance with another aspect of the invention, the processor is configured to execute additional software instructions for transmitting at least one tagged data element via the network interface. According to yet another aspect of the invention, the processor is configured to execute additional software instructions for upon tagging the at least one data element, inserting the at least one data element in the report template.

### DESCRIPTION OF THE FIGURES

FIG. 1 is a schematic block diagram of the electronic data delivery system for a physician's office in accordance with an exemplary embodiment of the present invention.

FIG. 2A is a frontal view of the data delivery device in accordance with an exemplary embodiment of the present invention.

FIG. 2B is a rear view of the data delivery device in accordance with an exemplary embodiment of the present invention.

FIG. 3 is an example of the web interface allowing remote access to the data delivery device in accordance with an exemplary embodiment of the present invention.

FIG. 4 is a schematic block diagram of the electronic data delivery system directed to insurance claim submission for a physician's office in accordance with an exemplary embodiment of the present invention.

FIG. 5 is a flowchart of the communication between a third party entity or host computer and the data delivery device located at a physician's office in accordance with an exemplary embodiment of the present invention.

### DESCRIPTION OF THE INVENTION

The present invention is directed to systems and methods for using a data delivery system and device used to transmit and receive electronic data for use at a physician's office over multiple types of secured communication channels between one or more third party entities including hospitals, doctor's offices, laboratories, pharmacies, vendors, insurance payers, pharmaceutical companies, transcription companies, etc. The communication channels may include fax, telephone, modem, Ethernet, WAN, LAN, as well as be capable of using a wide variety of networking protocols such as Internet Protocol, FTP, Telnet, TCP/IP, Point-to-Point Protocol (PPP), Challenge Handshake Authentication Protocol (CHAP), or other public or private networking protocols. The data delivery system provides for remotely accessing a data delivery device not only for transmitting and receiving data from a physician's office but also for maintenance, upkeep, troubleshooting and system auditing of the data delivery device.

In an exemplary embodiment of the present invention, the data delivery device is a small and easy to install hardware device that can be placed at a physician's office to facilitate the transmission and receipt of data and files to/from that physician office and to/from other third party entities (payers, pharmacies, labs, pharmaceutical companies, vendors, transcription companies, etc.). This device would also allow such communication activity to take place in the background without the need for direct real-time intervention by the office staff.

The data delivery device allows “push” as well as “pull” communications and instructions for activity that may take place in either the local office, or a remote “central” location. In certain embodiments of the present invention, the data delivery device may act as a remote application file server that can be leveraged by anyone wanting to send/receive data to/from a physician's office in a secure format. Entities seeking access to the physician office can “post” communications or instructions for access by the physician. The physician would control whether those remote parties may interact, and how such interaction may occur.

In an exemplary embodiment of the present invention, there are no interdependencies or interfaces required with local systems such as a physician office practice management system (POMIS), although interfaces may be used. The data delivery device also can facilitate both centralized and decentralized processing. “Dual” use of both direct-to payer and clearinghouse communication options with the data delivery device are linked to specific software. Further, the device can function as a host system's agent, the physician's agent, and/or a third party entity's agent. In an exemplary embodiment of the invention the data delivery device operates entirely behind a physician's firewall. The data delivery device may execute certain actions (e.g., submitting insurance claims files directly to an insurance payer system) from behind the physician's firewall allowing the device to function as a decentralized network. In addition, the device can support more than simply traditional and Health Insurance Portability and Accountability Act (HIPAA) standard transactions (e.g., lab results delivery).

In certain exemplary embodiments, the data delivery device may be configured to forward files of certain types to a host system for processing. This would allow the data delivery device to function as a facilitator to aggregate transactions into a central repository or processing center. It would also allow a physician direct, real-time control over access to clinical and financial data. In such a configuration with a host system, the data delivery system and device also provide for customization and/or personalization of services offered to a physician's office through the variation of system software modules and parameters (e.g., edits and filters). In exemplary embodiments, such customization of services may correspond to the class of user accessing the system or device. The classes of users may include the device manufacturers, system administrators, various levels of subscribing end user physicians, their staff members, or other medical professionals. Additionally, the functional capabilities of the data delivery device may be divided into various levels of accessibility for added security.

The present invention will be described below with reference to the accompanying drawings, in which exemplary embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The present invention is described below with reference to block diagrams of systems, methods, apparatuses and computer program products according to an embodiment of the invention. It will be understood that each block of the block diagrams, and combinations of blocks in the block diagrams, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create means for implementing the functionality of each block of the block diagrams, or combinations of blocks in the block diagrams discussed in detail in the descriptions below.

These computer program instructions may also be stored in a computer-readable memory that can direct a computer or other programmable data processing apparatus to function in a particular manner, such that the instructions stored in the computer-readable memory produce an article of manufacture including instruction means that implement the function specified in the block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the block or blocks.

Accordingly, blocks of the block diagrams support combinations of means for performing the specified functions, combinations of steps for performing the specified functions and program instruction means for performing the specified functions. It will also be understood that each block of the block diagrams, and combinations of blocks in the block diagrams, can be implemented by special purpose hardware-based computer systems that perform the specified functions or steps, or combinations of special purpose hardware and computer instructions.

The inventions may be implemented through an application program running on an operating system of a computer. The inventions also may be practiced with other computer system configurations, including hand-held devices, multiprocessor systems, microprocessor based or programmable consumer electronics, mini-computers, mainframe computers, etc.

Application programs that are components of the invention may include routines, programs, components, data structures, etc. that implement certain abstract data types, perform certain tasks, actions, or tasks. In a distributed computing environment, the application program (in whole or in part) may be located in local memory, or in other storage. In addition, or in the alternative, the application program (in whole or in part) may be located in remote memory or in storage to allow for the practice of the inventions where tasks are performed by remote processing devices linked through a communications network. Exemplary embodiments of the present invention will hereinafter be described with reference to the figures, in which like numerals indicate like elements throughout the several drawings.

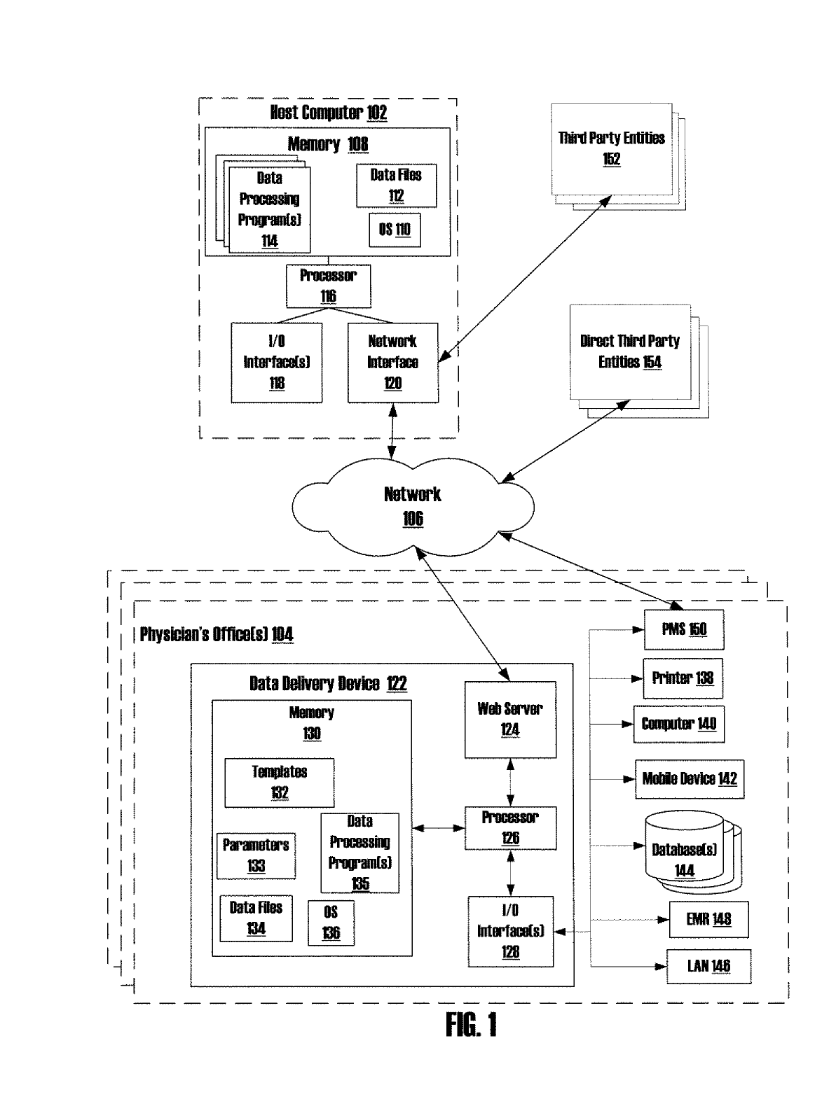

FIG. 1 is a schematic block diagram of the electronic data delivery system for a physician's office in accordance with an exemplary embodiment of the present invention. As shown in FIG. 1, a host computer 102 is in communication with a physician's office 104 through a network 106. The network 106 can be a dedicated private network including a LAN, WAN, T1 connection, or a public network such as the Internet. The network can also be one which supports any networking protocol including Internet Protocol, FTP, Telnet, TCP/IP, Point to Point Protocol (PPP), Challenge Handshake Authentication Protocol (CHAP), or other public or private networking protocol. In an exemplary embodiment of the present invention the network 106 is the Internet utilizing secured HTTPS protocol and user ID and password protected log-in security features. However, other secure methods of data transfer over public networks appreciable by one of ordinary skill in the art may also be used. The physician's office 104 may be any doctor's office, hospital, nursing home, pharmacy, laboratory, or any other similar healthcare facility.

According to FIG. 1, a host computer 102 (located remote from the physician's office 104 includes memory 108, a processor 116, I/O interfaces 118, as well as a network interface 120. In exemplary embodiments of the data delivery system, the host computer 102 communicates with the data delivery device located 122 at the physician's office 104 over the network 106. The host computer 102 may serve a variety of functions in the data delivery system. First, it may be a remote processing location for the processing or verification of data to be received at (or transmitted from) the data delivery device 122. For example, a physician's office 104 may desire to forward an insurance claim submission to a third party insurance payer. However, the physician's office 104 may have the claim information verified for accuracy or reviewed to see if the reimbursement has been optimized. While this verification functionality may be stored locally on the data delivery device 122, many of the data processing programs 114 handling the manipulation and/or verification of the submitted data may be centrally located at the host computer 102. By having these functions located at the host computer 102, the data delivery device 122 is simplified and allows for the option of a physician's office operator to select which verification and/or manipulation data processing programs 114 (e.g., software routines) to apply, or the physician's office 104 may subscribe to a particular set of data processing programs 114 to be run on particular data files submitted from the physicians office 104 to the host computer 102.

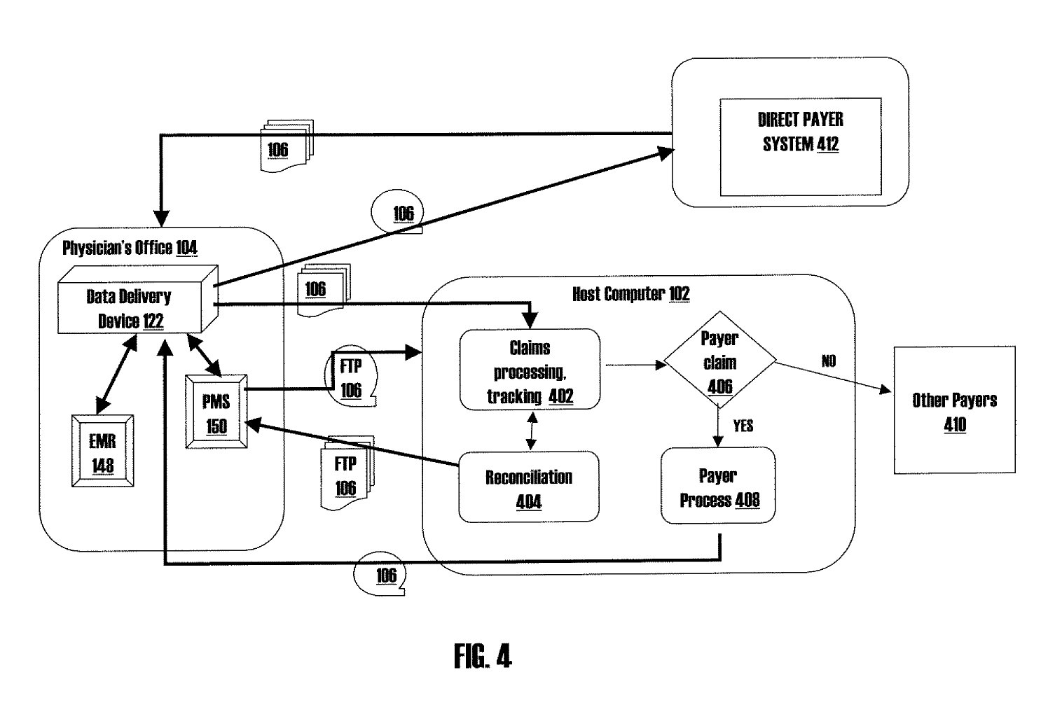

To perform data verification or manipulation, the processor 116 utilizes an operating system (OS) 110, which in turn calls data processing program 114 (located in the host computer's memory 108) to collect, manipulate, and/or format the data, contained in data files 112, so the data can be properly transmitted over the network 106 to a data delivery device 122 located at a remote location such as a physician's office 104 or transmitted to a third party entity 152. The manipulation or formatting of the data 112 to be transmitted may be as simple as tagging elements or sections of data for insertion into a report template, which can be completed by the data delivery device 122. Further, various data processing programs 114 may be selectively subscribed to by individual physician's offices 104. FIG. 4, described in further detail below, is an example of the types of data processing that may be conducted at the host computer 102.

Alternatively, the host computer 102 can simply act as a transmission portal such as a server or router, and the data processing program 114 functions could be done remote from the host computer 102 all together. Once the data has been properly formatted and/or tagged, the data could then be uploaded to the host computer 102 for transmission to a remote location over the network 106.

In yet another configuration, the host computer 102 may simply forward along information received from third party entities 152 to the data delivery device 122. In this configuration, the host computer 102 still may perform a variety of data manipulation, verification, translation, etc. prior to forwarding information from a third party entity 152 to the data delivery device 122 if the physician's office 104 desired such processing of the information. Again, such optional processing may be selectively subscribed to by individual physician offices 104. In the exemplary embodiment shown in FIG. 1, a direct third party entity 154 may bypass the host computer 102 entirely and communicate directly with the data delivery device 122 at a physician's office 104.

In an alternative embodiment of the present invention, additional manipulation, verification, or processing may be done with the information received from the data delivery device 122 at the host computer 102 through the I/O interfaces 118. This can be accomplished by manually entering the information through a keyboard, uploading the data from a disk drive, zip drive, USB port, CD-ROM/DVD-ROM drive, or uploaded through direct connection or network connection to other equipment or computing devices located in or remote from the host computer location. Finally, the data delivery device 122 may be configured to periodically contact the host computer 102 to request and/or receive software upgrades, reconfiguration, and/or troubleshooting.

The format of the data transmitted over the network 106 from the host computer 102 or direct third party entity 154 corresponds to the format or formats supported by a data delivery device 122 at the doctor's office 104, which is discussed in more detail below. These formats may include text files, Microsoft Word documents, Adobe Acrobat PDF files, TIFF (fax) files. ZIP files and other data formats appreciated by one of ordinary skill in the art. Once the data has been manipulated by the data processing programs 114 the data is then sent to the network interface 120 to be sent over the network 106 to the physician's office 104 or to the third party entity 152 depending on the ultimate destination of the data sent to the host computer 102.

In an exemplary embodiment of the present invention, the data delivery device 122 resides in a physician's office 104 behind a firewall associated with the physician's office 104. The data delivery device 122 may include a web server 124. Alternatively, the data can be delivered to the data delivery device 122 through the I/O interfaces 128 eliminating the need for receiving the data from a web server 124. However, the use of a dedicated on board web server 124 allows the data delivery device 122 to be remotely access through the Internet or some other network such as a private Intranet, LAN, WAN, T1 connection, or other networking configurations appreciable by one of ordinary skill in the art.

In an exemplary embodiment of the present invention, each data delivery device 122 has its own dedicated web server 124 providing for remote system monitoring and auditing over the network 106 (e.g., Internet). For example, a person with access to the network 106 may access the data delivery device 122 by accessing a secured web site through a web browser on a computer and entering a valid user identification and password, or satisfying various other methods of providing secured access. Rather than requiring a technically complex terminal emulator program to access a remote report printer as found in the prior art, any web browser (e.g., MS Internet Explorer, FireFox, Opera, Netscape, etc.) may be used to access the web server 124 of the data delivery device 122. Having connected to the device in this way, the user interface may provide a graphical interface that is continuously updated by updating files on the web server 124, as opposed to a teletype terminal.

In an exemplary embodiment of the present invention, the data delivery device 122 includes a web server 124, a processor 126, memory 130, and I/O interfaces 128. Once the data is received by the web server 124, the processor 126 utilizes the OS 136, which in turn utilizes a data processing program 135 to manipulate and/or control the delivered data, data files 134, report templates 132 and/or parameters 133 to determine if the data is valid and complete for its desired destination. The data processing program 135 may manipulate and perform other pre-processing of the data received from the host computer 102 or uploaded from the physician's office through the I/O interfaces 128. In an exemplary embodiment of the invention the OS 136 operating on the data delivery device is a “standard” software system (e.g., Linux) rather than a proprietary software system.

Once the data has been determined to have been correctly sent and checked that it has been completely delivered, the transmitted data may be stored in the memory 130 of the data delivery device 122 as a data file 134. In an exemplary embodiment, if the data sent requires no manipulation, extraction, or other processing by the data processing program 135, the data can be sent directly to the I/O interfaces 128 to be forwarded to a printer, display device, or other communication device, as will be discussed below. In an exemplary embodiment of the present invention, the received data (formatted, for example, as a Word document, PDF, etc.), which is accessible through the web server 124 or I/O interfaces 128 for extraction or manipulation, is also stored in the memory 130 of the data delivery device 122 for extended accessibility and auditing purposes.

Alternatively, the data may be sent as a text file (and associated images, where appropriate) or formatted as any file type by which the data processing program 135 can access and extract the data from in order to populate, for example, a pre-defined template 132. The templates 132 can be updated or customized through the web server 124 or I/O interface 128. To assist in the customization and manipulation of templates or stored data, the memory 130 may include numerous user-defined parameters 133 for use by the data processing program 135 (also referred to as the report processing program in some exemplary embodiments), which can be accessed and modified through the web server 124 or I/O interfaces 128 and provide customization options for the manufacturer, host computer 102 operator, or physician's office 104 operator. The parameters 133 can be used to automatically create reports that are customized specifically for a particular purpose every time a report is delivered.

Illustrative examples of the parameters 133 include types of data files that can populate a specific template 132 (e.g., text file, Word document, PDF, etc.), and where in the transmitted data file to look for particular information to appropriately populate the template 132. For example, column 1, row 1 of a text file may contain the patient's name, column two may contain doctor's office identification information, column three may contain data which corresponds to diagnosis codes or text description of symptoms, etc. The parameters 133 can be set through the web server 124 and/or I/O interface 128 to allow the data processing program 135 to determine what type of data file can populate a particular template 132 and what portion of that data file 134 corresponds to particular data to fill in a section of a template 132. Other forms of user defined parameters 133 as will be appreciated by one of ordinary skill in the art, include which colors or logos to establish on a template, what types of graphics are to be used in creating a report generated by the template, whether or not to include additional data such as patient information sheets (which provide boilerplate descriptions that correspond to the medical data contained in the transmitted data file to provide the patient with tangible take home information about their lab test results, diagnosis, symptoms, potential remedies, etc.). Parameters 133 can also be set to perform remote format conversion to and between Adobe Acrobat PDF files, TIFF (fax), HL7 (medical industry standard format), compressed ZIP files, etc.

It is further understood that a hierarchy of parameter 133 access can be established through various security measures including pass codes or log-in prompts including a user name, password, device serial number, etc. to provide different classes of users different levels of control over the parameters. For example, a manufacture may have the broadest access to set the parameters 133, the host computer would the have the next broadest level of access to the parameters 133 followed by the physician's office operator and so on. The parameters 133 can also be used not only to customize or manipulate delivered data, but to also customize the operation of the data delivery device 122 itself, particularly the I/O interfaces 128 and their respective operation, which will be discussed in more detail below. Examples of parameters 133 than can be manipulated to change the operation of the data delivery device 122 itself include changing the methods of device connectivity the particular data delivery device 122 will accept, as well as establishing the type of secured network connections, whether it will allow remote control access and control of the device itself, or allow particular types of networking or transfer protocol, which security features to enable/disable, etc.

This customization capability of the data delivery device 122 makes it possible to offload some of the functions traditionally performed by the host computer 102 or a remote server to the remote data delivery device 122. In particular, functions such as format conversion, print image rendering and graphic rendering can be performed on the data delivery device 122 itself. Thus, host computer 102 system functions can be “projected” out to the ends of the data delivery network—i.e., the clients' data delivery devices 122—rather than being performed by the server or at the host computer end of the network. There are a number of reasons that this “projection” of computing power may be desirable. First, the host computer 102 does not need to know or concern itself with the details of the equipment installed at the client site, rather it can confine itself to processing transmitted data in a simple, common format supported by all of the data delivery devices 122. The data delivery devices 122 will then take this “normalized” data and convert it as required by the client (e.g., physician's office) to produced the desired report. Second, because the data is transmitted in a simple, common format, it can be transmitted very quickly and inexpensively.

For example, a laboratory sending reports containing color graphics first generates the report (typically in Adobe Acrobat PDF format although other document formats can be supported such as Word, WordPerfect, or other document formats appreciable by one of ordinary skill in the art. Once then having determined (presumable from a database entry regarding the intended recipient) the make and model of printer that is installed at the client location, then converts the report to a printable image. This printable version of the file is typically ten to twenty times larger than the original and therefore takes that much longer to send. Of course a file that is ten times larger is ten times more prone to corruption.

By sending the original Adobe Acrobat PDF file to the data delivery device 122 and letting it do the print formatting, the laboratory no longer needs to know what type of printer is maintained by each client, no longer needs to convert it to the much larger print image and no longer needs to take all that extra time or expense to send it. Further, with a copy of the original report now at the client site, it can be converted into several new formats for printing, viewing via a web browser, for sharing over a local network, or other functions appreciable by one of ordinary skill in the art. Once the transmitted data file has been manipulated and/or extracted for populating a template by the data processing program 135 and thus create a report, the report can then be sent to the web server 124 or I/O interfaces 128 either automatically or upon receiving a command to do so from the web server 124 and/or I/O interfaces 128.

The I/O interfaces 128 can support a wide variety of connectivity means, each individually appreciable by one of ordinary skill in the art such as serial ports, parallel ports, phone jacks, Ethernet jacks, 802.11x wireless networking card slots, USB ports, Bluetooth antenna, etc. Such a wide variety of connectivity supported by the data delivery device 122 allows for connectivity options to a wide variety of equipment, and including other communication devices located in or remote to the physician's office 104. Files can be transferred by FTP, HTTP, HTTPS, Telnet, SSL and other networking protocols appreciable by one of ordinary skill in the art. Each provides additional security, error free transmission, and far greater speed than teletype transmission. Reports or data files generated by the host computer 102 or the data delivery device 122, itself, can now be transferred to a remote device simply, quickly, and the accuracy of the transmitted reports is immediately verifiable or automatically verified.

As shown in FIG. 1, the devices that can be in communication with data delivery device 122 include printers 138, computers 140, mobile devices 142 such as cell phones, blackberries, PDA's, etc., databases 144 remote to the data delivery device 122 connectivity to an existing LAN 146, security devices 148 such as USB security keys, as well as other equipment and communication devices appreciable by one of ordinary skill in the art. Additionally, the data delivery device 122 may be in communication with the physician office's practice management system (PMS) 150 and/or its associated electronic medical records system (EMR) 148. Because the data delivery device 122 may have access to the data located in both the PMS 150 and EMR 148, the host computer or other third party entities may have access to retrieve or update data located in either system. Further, software upgrades and troubleshooting of either the PMS 150 or EMR 148 may also be conducted remotely through the data delivery device 122.

Further, in an exemplary embodiment of the invention the data delivery device 122, through utilization of its own dedicated web server 124 and public Internet network connection capability, may be accessible by a user remote or local to the physician's office 104 through a web browser through a secure means such as HTTPS protocol requiring user name and password to access. The user can control and troubleshoot devices the I/O interfaces 128 of the data delivery device 122 are attached to.

Networking also allows the data delivery device 122 to interact with the end user in new and different ways. For example, a physician can now access the data delivery device 122 to access received data using his web browser. The data delivery device 122 can also be configured to print received reports on an existing network printer in some other part of the building/world. It is even possible to specify that some reports should print on one printer and others should print on another. Such activity may be logged in the memory 130 and also accessible through a web browser for confirmation and troubleshooting capabilities. Further, should the printer produce an error or fail to print, messages (such as toner low, paper jam, etc.) can be relayed from the printer to a remote user accessing the web server 124 via a web browser. Moreover, diagnostic checks or even commands from a web browser to the printer may be sent to the printer via the I/O interfaces 128 of the data delivery device 122. Additionally reports may be converted to various formats and sent to a doctor's PDA, cell phone or other mobile communication device 142, or to a computer or computer network remote from the office or hospital, to a doctor's dedicated webpage, email address or electronic delivery means.

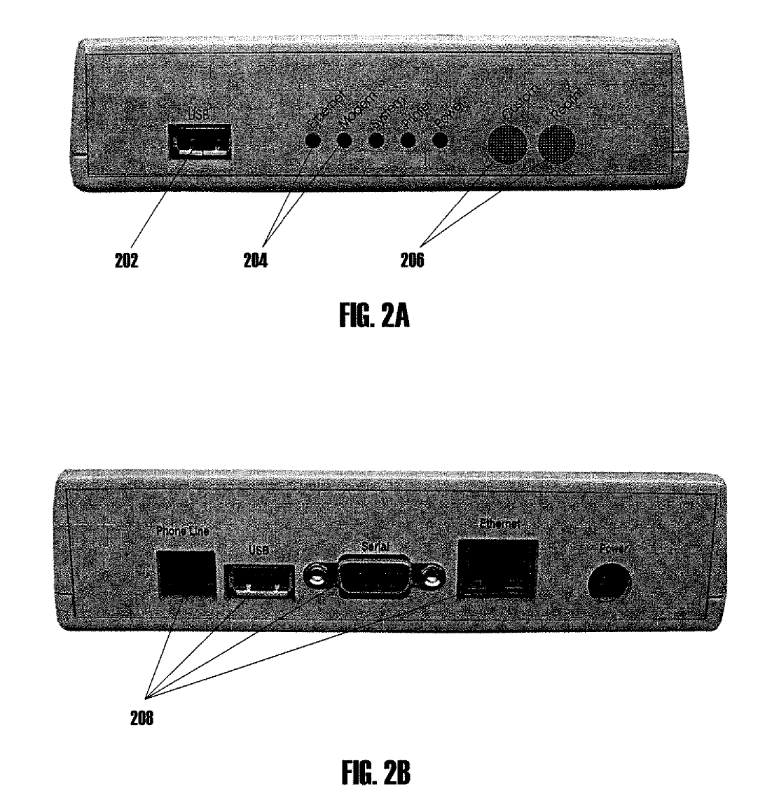

FIGS. 2A and 2B are frontal and rear views of a data delivery device 122, respectively, in accordance with an exemplary embodiment of the present invention. As shown in FIG. 2A, a USB interface 202 is part of the I/O interface 128 on the data delivery device 122, and allows the data delivery device 122 to connect to any device or added feature such as portable memory sticks (or other external memory devices), hand held computer synchronization, and data transfer. USB interfaces also can provide support for printers, Bluetooth interfaces, WiFi interfaces, encrypted security keys, software updates from a memory key, as well as other devices capable of communicating through a USB interface. The front view of the data delivery device 122 shows an LED display 204 with indicator lights to allow a user to know what particular operation the data delivery device 122 is undertaking and to allow additional troubleshooting of the device itself or a device in communication with the data delivery device 122.

Also on the front of the device are buttons 206. These buttons 206 provide another user means for communicating with the data delivery device 122 and inputting commands to the device to perform operations including reprinting a report, forwarding a report on to a particular location, halting a transmission, resetting the device to a particular pre-set state. As one of ordinary skill in the art will appreciate, this on-box user interface may be expanded to include one or more LCD displays, and/or displays incorporating touch screen technology to provide further user interaction at the physical device itself to provide all or some of the functionality a user has through a web browser, as discussed with reference to FIG. 1 above.

As shown in FIG. 2B, various connectivity options 208 are supported by the device such as serial ports, phone jacks, Ethernet jacks, USB ports as well as other connectivity options not shown in FIG. 2B. These connection points allow for the functioning of the I/O interface 128 discussed with reference to FIG. 1.

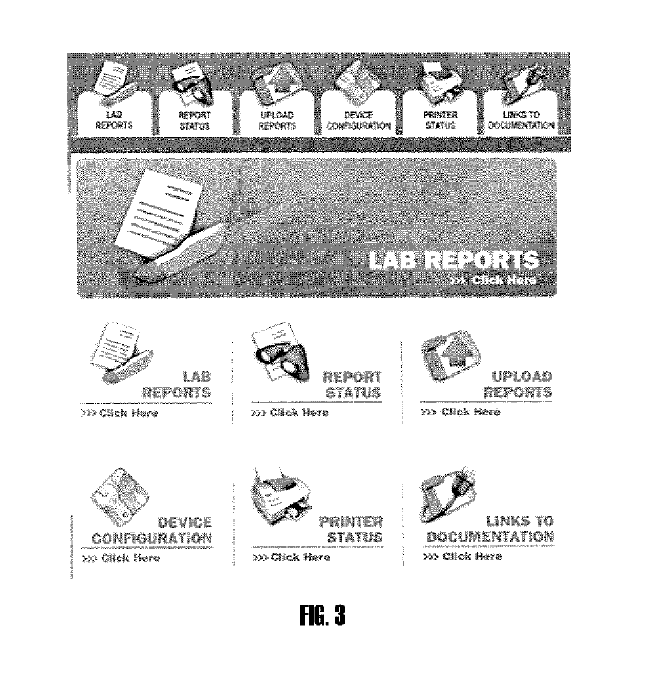

FIG. 3 is an example of the web interface allowing remote access to the data delivery device 122 in accordance with an exemplary embodiment of the present invention. In an exemplary embodiment of the invention, this webpage would only be accessible after the submission of a user name and password over a secured network protocol such as HTTPS or other forms of encryption techniques appreciable by one of ordinary skill in the art. The user can select what interaction the user wants with the data delivery device by selecting an icon corresponding to that type of device interaction. In FIG. 3, the types of interaction icons shown include Report Status, Upload Reports, Device Configuration, Printer Status, and Links to Documentation. However, other functionality discussed above with reference to FIG. 1 can also be supported on this web interface.

Selecting the Report Status icon allows a user to view the status and access reports or data files that the data delivery device 122 has received and stored in its memory 130. This page can be set to refresh automatically after a certain time period to make verification that a report or data file has been successfully delivered an easier process. By accessing the report, which may require an additional level password protection, a user can make adjustments to the look and feel of the application as well as the data reported itself (correct typos, incorrect information, etc.).

Selecting the Upload Reports icon allows a user to upload a report or data file to the data delivery device 122. Whereas selecting the Device Configuration icon allows a user to access the customizable parameters which establish how the device operates as discussed above with reference to FIG. 1. This icon may require additional password or other security clearance information to access a variety of parameter that may or may not be restricted depending on the identity the user. Selecting the Printer Status icon allows a user to view or troubleshoot the printer device that is connected to the delivery device. This allows the user to see if a report has printed, failed to print, what caused the failure, if the print is on-line, etc. It also can allow the printer to communicate with the printer though command prompts. Selecting the Links to Documentation icon allows a user to access documentation relating to the functionality and operation of the data delivery device 122 as well as links to help desk and corporate web sites.

FIG. 4 is an exemplary embodiment of the delivery device setup with several options for submitting insurance claims to a payer system. As shown in FIG. 4, a physician's office 106 is in communication with a host computer 102 and a direct payer system 412 over a network 106. In an exemplary embodiment of the invention, the physician's office 104 communicates with the both the direct payer 412 and the host computer 102, although the practice management system (PMS) 150 may communicate billing information directly with the host computer 102 for updated information or as another way to communicate with the host computer 102 should the data delivery device 122 be rendered inoperative or non-responsive. In the exemplary embodiment shown in FIG. 4 the PMS 150 and host computer communicate by FTP, however, other communication methods as appreciable by one of ordinary skill in the art may also be used. At the physician's office 104, the data delivery device 122 is in communication with both the PMS 150 and the electronic medical record system (EMR) 148. Thus, the data delivery device may send and retrieve information from the PMS 150 or EMR 148 systems. For example, the data delivery device 122 may obtain claim information from the PMS 150 relating to a patient of the physician's office 106. The claim information may be a complete claim submission which the data delivery device 122 may submit directly to a direct payer system 412 for processing. Alternatively, a complete claim submission or simply claim data may be transmitted to the host computer 102 where the data may be verified and/or manipulated. For example, claim data relating to specific patient-related information such as name, address, an insurance policy number, corresponding procedure code, etc. may be forwarded to the host computer 102 where it may be assembled into a properly formatted claim and forwarded along to a payer system 410.

In an exemplary embodiment shown in FIG. 4, the data delivery device 122 may submit a claim to the host computer 102 over the network 106. In sending the claim information, the data delivery device 122 may be commanded to “push” the claim data to the host computer 102 by an operator accessing a user interface located on a secured website (e.g., HTTPS) or LAN address location. Alternatively, the data delivery device 122 may be configured to automatically contact the host computer at periodic intervals. When connected, the host computer 102 may “pull” data from the data delivery device 122 as well as perform other operations such as transmit new information to the data delivery device 122 to be used at the physician's office 104. Alternatively, the host computer 102 may perform some upgrading, troubleshooting, or various maintenance procedures for the data delivery device 122 and even the PMS 150 or EMR 148 that are in communication with the data delivery device 122.

Once the claim has been sent to the host computer 102, the claim may undergo a variety of processing steps (e.g., edits, filters, etc.). An exemplary process conducted on a claim submission at the host computer 102 is shown in FIG. 4 starting at claim processing/tracking 402, where the host computer 102 logs the claim submission locally and runs the claim submission through its reconciliation software routines 404 (e.g., edits, filters, etc.) which provide a variety of checks to the submitted claim. For example, the claim may be checked for all fields being properly filled, spelling, accurate patient information, correct procedure codes, etc.

Other routines may be employed to conduct further verification and/or optimization to ensure that the maximum reimbursement allowed is being requested, etc. In an exemplary embodiment of the invention, the reconciliation routines 404 (e.g., edits or filters, etc.) may be subscription based where a physician's office 104 would select which reconciliation routines 404 they would like to have run on their claim submissions. The host computer 102 may also handle the claim submissions for a particular payer 406. If so, then the claim is sent to that particular payer to be processed 408 and then the processed claim is sent back to the corresponding physician's office's data delivery system 122. Alternatively, the host computer 102 may simply forward a claim submission to another payer 410, or perform its reconciliation edits and filters 404 and then forward the claim submission to the other payer 410. That payer 410 may communicate back to the host computer 102 which will forward the claim submission response to the corresponding data delivery device 122, or alternatively, the other payer 410 may send the claim submission response to the corresponding data delivery device 122 directly (although it is not shown in the embodiment of FIG. 4).

While the embodiment shown in FIG. 4, demonstrates the claim submission process, similar configurations and processes may be conducted when the data delivery device 122 of the physician's office 104 wants to communicate prescription information to pharmacy, inventory and ordering information to vendors or pharmaceutical companies, patient medical data and/or records to laboratories or other doctor offices, as well as other electronic data transferring often required during the course of business at a physician's office 104.

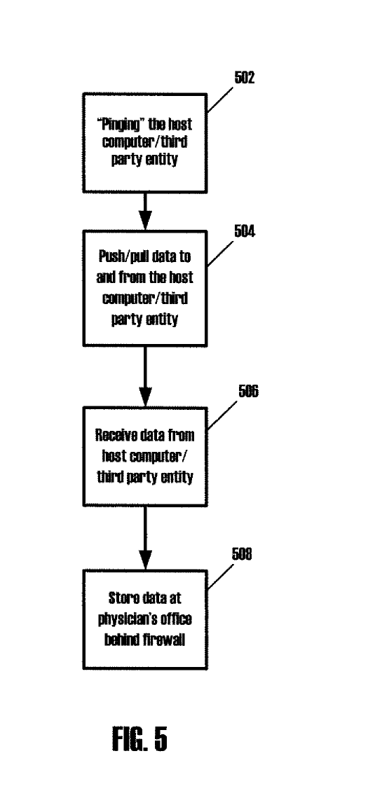

FIG. 5 is a flowchart of the communication between the host computer and the data delivery device located at a physician's office in accordance with an exemplary embodiment of the present invention. The communication beings at step 502 where the data delivery device “pings” the host computer at a periodic interval. Once the data delivery device is in communication with the host computer, step 504 is invoked and the host computer “pulls” (or receives) any information that is available at the physician's office to the host computer In an exemplary embodiment of the invention, the data delivery device is at all times virtually accessible from the remote central location for update and configuration changes, as it will be programmed to reach “home” on regular intervals to check for updates, new executables, etc.

Alternatively, the data delivery device may be configured to automatically “push” (or transmit) its data to be sent to the host computer without waiting for a request (or “pull”) for such data sent from the host computer. The data delivery device may be configured to transmit data to the host computer when commanded to do so by an operator located either at or remote from the doctor's office.

In yet another alternative embodiment, the data delivery device may “push” (or transmit) data directly to a third party entity, bypassing the host computer, or, alternatively, a third party may “pull” (or request) the information from the data delivery device. Once the data delivery device has transmitted its data to the host computer or third party entity it waits to receive data at step 506 from either the host computer or the third party entity. However, it will be appreciated by one of ordinary skill in the art, that the data delivery device may transmit or receive data in any order and even simultaneously through the use of two communication interfaces.

When the data delivery device receives data to be used at the physician's office, step 508 is invoked and the data delivery device stores the received data in a storage location (e.g., database, PMS, EMR, etc.) that is accessible on the physician's office LAN or another location easily accessible at the physician's office. The data may be stored behind a firewall associated with the physician's office for added security. In an exemplary embodiment, multiple transmissions received at the data delivery device will be stored at the same accessible location to consolidate the electronic data received at the physician's office for easy access and management of the received data.

In an alternative embodiment, the data delivery device may be configured to receive data files and execute any of a number of given sets of instructions for those files. Examples of such executable instructions include (1) direct file submission to a payer from the physician's office, (2) automatic printing of lab reports to a LAN printer, (3) automatic publishing of patient demographic edits to a local network file to later be picked up by the practice's POMIS, (4) pulling don lab result reports from a host computer or a Lab Information System (LIS) and posting those reports to a physician's EMR. Other executable instructions appreciable by one of ordinary skill in the art also may be conducted by the data delivery system upon receiving the data file(s) containing such executable instructions.

In an exemplary embodiment of the present invention, the data delivery device and the storage locations of its received data are accessible through a secured user interface where the data may be accessed, manipulated, and/or moved to another location. Additionally, the user interface will allow a physician office operator to command the data delivery device to transmit a particular file or data set to the host computer or third party entity either upon execution of a send command or at the next periodic interval when the data delivery device “pings” the host computer or third party entity.

Accordingly, many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of communicating with a physician's office, comprising:
receiving, via a network interface, at least one data file from a data delivery device located in a physician's office;
determining a set of data processing programs from a plurality of stored data processing programs to be utilized when processing the at least one data file, wherein the determination is made based on a subscription associated with the physician's office; and
processing the at least one data file utilizing at least a portion of the determined set of data processing programs, wherein the processing includes at least one of collecting and manipulating the at least one data file.

2. The method of claim 1, further comprising:
receiving, via at least one input/output (I/O) interface, additional data files.

3. The method of claim 1, further comprising:
transmitting, via the network interface, reconfiguration data to the data delivery device.

4. The method of claim 1, wherein manipulating the at least one data file includes formatting the at least one data file.

5. The method of claim 1, wherein manipulating the at least one data file includes tagging at least one data element for insertion into a report template.

6. The method of claim 5, further comprising:
transmitting at least one tagged data element via the network interface.

7. The method of claims 5, further comprising:
upon tagging the at least one data element, inserting the at least one data element in the report template.

8. A data delivery device associated with a physician's office comprising:
a memory, wherein the memory contains at least one data processing program and a plurality of report templates;
a web server connected to a network, wherein the web server is accessible via a web browser, and wherein the web server is located behind a firewall at a physician's office;
a processor, in communication with the memory and web server, wherein the processor is configured to execute software instructions for:
receiving at least one data file via the web browser,
reformatting the at least one data file to a format readable by the at least one data processing program, and
processing the at least one data file utilizing the at least one processing program, wherein the processing includes extracting data from the at least one data file and populating at least one report template with at least a portion of the extracted data.

9. The data delivery device of claim 8, wherein the processor is configured to execute additional software instructions for:
storing the received data file in the memory.

10. The data delivery device of claim 8, wherein the processor is configured to execute additional software instructions for:
transmitting the at least one report template to the web server.

11. The data delivery device of claim 8, wherein the memory includes a plurality of user-defined parameters, and wherein the processor is configured to execute additional software instructions for:
customizing the at least one report template based at least in part on the plurality of user-defined parameters.

12. The data delivery device of claim 8, further comprising at least one input/output (I/O) interface in communication with the processor, wherein the processor is configured to execute additional software instructions for:
transmitting the at least one report template to an external device.

13. The data delivery device of claim 8, further comprising at least one input/output (I/O) interface in communication with the processor, wherein the at least one I/O interface is in communication with at least one of a practice management system associated with the physician's office and an electronic medical records system associated with the physician's office.

14. A host computer comprising:
a memory, wherein the memory contains a plurality data processing programs;
a network interface in communication with a network; and
a processor, in communication with the memory and network interface, wherein the processor is configured to execute software instructions for:
receiving, via the network interface, at least one data file from a data delivery device located in a physician's office remote from the host computer,
determining a set of the plurality of data processing programs to be utilized when processing the at least one data file, wherein the determination is made based on a subscription associated with the physician's office, and
processing the at least one data file utilizing at least a portion of the determined set of data processing programs, wherein the processing includes at least one of collecting and manipulating the at least one data file.

15. The host computer of claim 14, further comprising at least one input/output (I/O) interface in communication with the processor, wherein the processor is configured to execute additional software instructions for:
receiving, via the at least one I/O interface, additional data files.

16. The host computer of claim 14, wherein the processor is configured to execute additional software instructions for:
transmitting, via the network interface, reconfiguration data to the data delivery device.

17. The host computer of claim 14, wherein the software instructions for manipulating the at least one data file include formatting the at least one data file.

18. The host computer of claim 14, wherein the software instructions for manipulating the at least one data file include tagging at least one data element for insertion into a report template.

19. The host computer of claim 18, wherein the processor is configured to execute additional software instructions for:
transmitting at least one tagged data element via the network interface.

20. The host computer of claim 18, wherein the processor is configured to execute additional software instructions for:
upon tagging the at least one data element, inserting the at least one data element in the report template.
